(19) United States
(12) Patent Application Publication
(10) Pub. No.: US 2024/0374559 A1
(43) Pub. Date: Nov. 14, 2024

(54) COMPOSITION COMPRISING PPAR-MODULATOR AND AN UROLITHIN DERIVATIVE AND USES THEREOF

(71) Applicants: Université de Bordeaux, BORDEAUX (FR); Centre Hospitalier Universitaire de Bordeaux, TALENCE cedex (FR); Institut National de la Santé et de la Recherche Médicale - INSERM, Paris (FR)

(72) Inventors: Rodrigue ROSSIGNOL, PESSAC (FR); Laetitia DARD, MERIGNAC (FR); Didier LACOMBE, FLOIRAC (FR); Nivea DIAS AMOEDO, PESSAC (FR)

(21) Appl. No.: 18/559,020

(22) PCT Filed: Jan. 21, 2022
(86) PCT No.: PCT/EP2022/051382

§ 371 Date: Nov. 3, 2023

(30) Foreign Application Priority Data
May 6, 2021 (EP) 21305586.6

(51) Int. Cl. A61K 31/37; A61K 31/195; A61K 31/216; A61P 9/00

(52) U.S. Cl. CPC A61K 31/37; A61K 31/195; A61K 31/216; A61P 9/00

(57) ABSTRACT

The present invention belongs to the field of medicine, and more precisely to the field of medicine useful in the treatment of cardiac diseases with contributing mitochondrial dysfunction.

The present invention relates to a composition a pharmaceutical composition comprising a PPAR-modulator and an urolithin derivative of formula I:

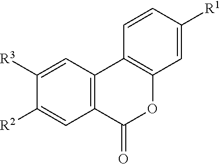

wherein R¹, R² and R³ independently represents H or OH and its use thereof in the treatment of various cardiac diseases linked to cardiac diseases with contributing mitochondrial dysfunction.

### TECHNICAL FIELD

The present invention belongs to the field of medicine, and more precisely to the field of medicine useful in the treatment of cardiac diseases caused by energy metabolism dysfunction.

The present invention relates to a pharmaceutical composition comprising a PPAR-modulator and an urolithin derivative and its use thereof in the treatment of various cardiac diseases linked to cardiac diseases with contributing mitochondrial dysfunction, such as the Costello syndrome and the Barth syndrome for instance.

### TECHNICAL BACKGROUND

According to the American Center for Diseases Control and Prevention, heart disease is the leading cause of death for men, women, and people of most racial and ethnic groups in the United States. One person dies every 36 seconds in the United States from cardiovascular disease and about 655,000 Americans die from heart disease each year—that's 1 in every 4 deaths. Heart disease costs the United States about $219 billion each year from 2014 to 2015. This includes the cost of health care services, medicines, and lost productivity due to death.

Mitochondria have emerged as a central factor in the pathogenesis and progression of heart failure, and other cardiovascular diseases, as well, but no therapies are available to prevent, to reduce or to treat mitochondrial dysfunction. The National Heart, Lung, and Blood Institute convened a group of leading experts in heart failure, cardiovascular diseases, and mitochondria research in August 2018 [1]. These experts concluded that mitochondrial dysfunction and energy deficiency are strongly implicated in the development of hypertrophic cardiomyopathy (HCM) and of heart failure [2]. Omics studies performed on patients with HCM suggested that perturbed metabolic signalling and mitochondrial dysfunction are common pathogenic mechanisms in patients with HCM of diverse genetic origins, highlighting the possibility to attenuate the clinical disease through improving metabolic function and reducing mitochondrial injury [3].

Despite the use of guideline-directed therapies, the morbidity and mortality of heart failure remains unacceptably high. Novel therapeutic approaches are thus greatly needed. In this context, targeting mitochondrial dysfunction and energy metabolism in heart disease may provide novel approaches for the prevention and the treatment of cardiac diseases. Moreover, as mitochondrial dysfunction including defective fatty acid oxidation, abnormal mitochondrial structure, respiratory dysfunction, and failure to upregulate mitophagic clearance was detected at early stage in the progression of HCM of diverse genetic origins [3], HCM therapeutics targeting these metabolic alterations and mitochondrial dysfunction could be proposed at early stage of the disease. The causes of heart failure are numerous and include, for instance, coronary artery disease (atherosclerosis), past heart attack (myocardial infarction), high blood pressure (hypertension or HBP), abnormal heart valves, heart muscle disease (dilated cardiomyopathy, hypertrophic cardiomyopathy) or inflammation (myocarditis), severe lung disease, diabetes, obesity, sleep apnea and heart defects present at birth (congenital heart disease). A large number of rare genetic defects are responsible for cardiac disease and heart failure, among other symptoms. Such pathologies are for instance the mitochondrial diseases, or RASopathies.

Germline mutations that activate RAS/MAPK signaling are responsible for the ‘RASopathies’, a group of rare human developmental diseases that affect more than 400,000 individuals in the United States alone [4]. Costello syndrome (CS) was the first RASopathy discovered in 1971 [5] and was described as a multiple congenital anomaly syndrome caused by heterozygous activating germline mutations in HRAS. Most individuals affected by CS carry a mutation in HRAS at the G12 position, and more than 80% of CS individuals have a p.G12S substitution. The second most-common substitution is p.G12A [6-7]. These mutations maintain HRAS in a constitutively (over) active state [8]. Because of the RAS/MAPK pathway activation, development is altered and most children with CS exhibit an increased birth weight, dysmorphic craniofacial features, failure to thrive and gastro-esophageal reflux with oral aversion, especially during the newborn period. CS can also involve the skin with excessive wrinkling and redundancy over the dorsum of the hands and feet, along with deep plantar and palmar creases [9], as well as an increased risk of developing benign or malignant tumors [9-11].

A central feature of CS and other RASopathies is hypertrophic cardiomyopathy (HCM) [12-13]. Musculoskeletal abnormalities such as hypotonia were also reported in CS. Furthermore, HCM was observed in transgenic mutant HRAS mouse models or in patients expressing constitutively active forms of HRAS [14-19], but the molecular mechanisms linking HRAS activation with cardiac or skeletal muscle dysfunction remain unknown [20-21]. However, cardiac involvement remains a major determinant for the prognosis of CS [22], raising the need to propose adapted therapeutic strategies able to improve the quality of life of these patients and reduce complications. Several lines of evidence have indicated that HCM typically associates with energetic insufficiency caused by bioenergetic alterations of the heart [23-24]. In particular, genetic, environmental or age-related defects in oxidative phosphorylation (OXPHOS) and fatty oxidation (FAO) machineries can lead to HCM [25]. Integrated Omic studies also showed that mitochondrial dysfunction including defective fatty acid oxidation, abnormal mitochondrial structure, respiratory dysfunction, and failure to upregulate mitophagic clearance was detected at early stage during the progression of HCM of diverse genetic origins [3]. Hypertrophic cardiomyopathy (HCM) is a heterogeneous cardiac disease with a diverse clinical presentation and course. HCM is a genetic disorder with the prevalence of 1/500 globally, with an incidence of sudden cardiac death (SCD) or sudden cardiac arrest (SCA) of 0.5-1% per year. It is also a common inherited heart disease with serious adverse outcomes, including heart failure, arrhythmias, and sudden cardiac death.

According to the American Heart Association, ‘alterations in mitochondrial function are increasingly being recognized as a contributing factor in myocardial infarction and in patients presenting with cardiomyopathy’ [26]. Therefore, we hypothesized that CS could include early alterations of heart bioenergetics and mitochondrial physiology participating to the development of HCM. This hypothesis was also based on a prospective screening of 18 patients with various Ras-MAPK pathway defects that detected biochemical signs of disturbed OXPHOS [27], and on the partial overlap of clinical manifestations between mitochondrial diseases and RASopathies [28].

Heart bioenergetics strongly relies on OXPHOS, and the heart-beating function is linearly dependent on the flux of mitochondrial ATP synthesis [29-30]. Moreover, the regulation of mitochondrial turnover, including organelle biogenesis and degradation, is a major site of bioenergetic control in heart physiology [31-32]. Mitochondrial biogenesis requires the coordinated expression of a set of 1,136 genes controlled, to a large extent, by the master regulator 5′AMP-activated protein kinase (AMPK) [33]. Briefly, AMPK stimulation induces activation of peroxisome proliferator-activated receptor gamma coactivator 1-alpha (PGC1α), a transcriptional co-activator that promotes mitochondrial biogenesis in cooperation with specific transcription factors [34]. AMPK activation also promotes mitochondrial degradation in conjunction with organelle biogenesis, resulting in a net increase in mitochondrial quality and function [35-36]. The central role of AMPK and mitochondrial turnover in HCM pathophysiology was demonstrated in vivo using transgenic mouse knock-out for AMPKα2 [37], PGC1a [31-32], NRF2 [38], ERRα or PPARα [40]. All mouse models developed HCM (at rest or under stress) caused by defective mitochondrial turnover, quality control and bioenergetics. Finally, mitochondrial biogenesis is a druggable process, and pharmacological activators of AMPK or PGC1a prevent HCM development in preclinical studies [41-44].

Yet, there is no treatment available in the clinics to stimulate mitochondrial and cytosolic energy metabolism in the heart and to rescue a reduction in mitochondrial content and/or a defective organelle turnover and quality control. There is a major unmet need to prevent and rescue mitochondrial dysfunction for the prevention and treatment of cardiac disease in RASopathies and mitochondrial diseases of genetic or chemical origin [1]. There is therefore a need for a composition that solves the above listed problems.

### DETAILED DESCRIPTION OF THE INVENTION

Applicant has developed new pharmaceutical composition, comprising a combination of compounds that may be used as treatment to stimulate mitochondrial turnover and energy metabolism in the heart, and other tissues as well, and to rescue a reduction in mitochondrial efficacy and/or a defective organelle bioenergetics, biogenesis, turnover, dynamics, proteostasis and quality control. The composition according to the invention therefore meets the need to prevent and rescue mitochondrial dysfunction for the prevention and treatment of cardiac disease not only in RASopathies and mitochondrial diseases, but also in cardiopathies with a contributing bioenergetic deficiency resulting from mitochondrial alterations in organelle composition, turnover, quality control, proteostasis, signalling, dynamics, fueling and efficacy and REDOX homeostasis. The composition according to the invention therefore solves partially or completely the problems listed above.

The composition according to the invention comprises PPAR-modulator and an urolithin derivative.

The composition according to the invention has proven useful as a medicine, and in particular in the treatment of cardiac diseases with contributing mitochondrial dysfunction.

Thus, an object of the invention is a pharmaceutical composition comprising a PPAR-modulator and

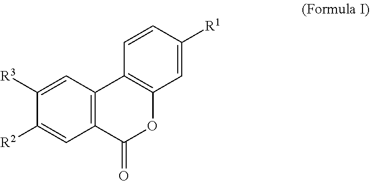

an urolithin derivative of formula I:
wherein R¹, R² and R³ independently represents H or OH.

It is meant herein by an “urolithin derivative of formula I”, a “compound of formula I”. Advantageously, the urolithin derivative of formula I may be urolithin A (R¹ and R²═OH and R³═H), B (R¹═OH and R² and R³═H) and/or C (R¹, R² and R³═OH):

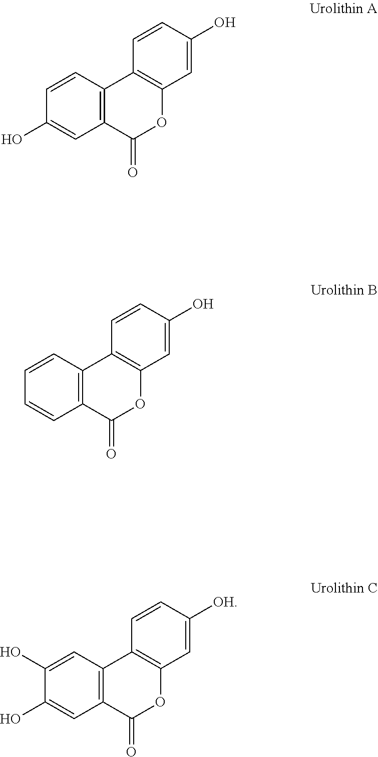 [SMILES: O=c1oc2cc(O)ccc2c2ccc(O)cc12 ; O=c1oc2cc(O)ccc2c2ccccc12 ; O=c1oc2cc(O)ccc2c2cc(O)c(O)cc12]

The urolithin derivative of formula I may preferably be urolithin A.

In the context of the invention, it is meant by “PPAR-modulator”, an agonist of any of or all of the different PPAR receptors.

Advantageously, the PPAR modulator may be chosen in the group comprising PPAR alpha agonists, PPAR gamma agonists, PPAR delta agonists, PPAR dual agonists (alpha/gamma or alpha/delta) and PPAR pan agonists (alpha/gamma/delta). The PPAR modulator may be selected from PPAR alpha agonists, PPAR gamma agonists, PPAR delta agonists, PPAR dual agonists (alpha/gamma or alpha/delta) and PPAR pan agonists (alpha/gamma/delta).

It is meant by “PPAR alpha agonists”, a stimulator of PPAR alpha receptors-mediated signalling pathway.

It is meant by “PPAR gamma agonists”, a stimulator of PPAR gamma receptors-mediated signalling pathway.

It is meant by “PPAR delta agonists”, a stimulator of PPAR delta receptors-mediated signalling pathway.

Advantageously, the PPAR-modulator may be selected from any one or more from table 1:

TABLE 1
Example of PPAR-modulators according to the invention:

PPAR alpha agonists
Approved drugs - fibrates: fenofibrate, clofibrate, gemfibrozil,
ciprofibrate, bezafibrate, Eupatilin (NSC 122413), Raspberry ketone
PPAR gamma agonists
drug class of thiazolidinediones; Actos (pioglitazone), Avandia
(rosiglitazone), Pseudoginsenoside F11, Glabridin, MRL24, Leriglitazone
PPAR delta agonists
Cardarine (GW501516), L165041, GW0742
PPAR dual agonists (alpha/gamma)
Muraglitazar, Tesaglitazar, Ragaglitazar, Naveglitazar, GW0742,
PPAR pan agonists (alpha/delta/gamma)
Lanifibranor (IVA-337), Bavachinin (7-O-Methylbavachin)
PPAR dual agonists (alpha/delta)
Elafibranor (GFT505)

Advantageously, the PPAR-modulator may be any of the PPAR-modulator described in the review from Ichiro Takada & Makoto Makishima (2020) [45], the content of which is included by reference. Thus, the PPAR-modulator may be chosen in the group comprising fenofibrate; bezafibrate; L165041; GW0742; GW501516; pioglitazone; rosiglitazone; MRL24; 9-Hydroxy-10(E), 12(E)-octadecadienoic acid; ethyl 4-hydroxy-3,3-methylpentanoate; LY518674; hexadecanamide; palmitoylethanolamide; 9-octadecenamide; GW7647; DY121; TZD salt; 5-Hydroxy-4-phenylbutenolide; benzoate; phenylacetate; MTTB; INT131; LDT477; saroglitazar; elafibranor; lanifibranor; and may be selected from any of the compounds the FIG. 3 from the above mentioned article of Ichiro Takada & Makoto Makishima (2020) [44].

Advantageously, the PPAR-modulator may preferably be selected from PPAR alpha agonists, and more preferably from fibrates. The PPAR-modulator may preferably be bezafibrate or fenofibrate, and more preferably bezafibrate.

Bezafibrate is also known as 2-(4-{2-[(4-chlorobenzoyl)amino]ethyl}phenoxy)-2-methylpropanoic acid (CAS 41859-67-0). Bezafibrate is marketed under the brand name Bezalip® (and various other brand names). Bezafibrate is a fibrate drug that may be used as a lipid-lowering agent to treat hyperlipidaemia in adults and children. It is also known to help to lower LDL cholesterol and triglyceride in the blood and increase HDL.

Fenofibrate is also known as propan-2-yl 2-[4-(4-chlorobenzoyl) phenoxy]-2-methylpropanoate (49562-28-9). Fenofibrate, is marketed under various brand names such as Tricor, Fenoglide or Lipofen for example. Fenofibrate is a medication of the fibrate class used to treat abnormal blood lipid levels.

L165041 is also known as 4-[3-(4-Acetyl-3-hydroxy-2-propylphenoxy) propoxy]phenoxy-acetic acid (CAS 79558-09-1).

GW0742 is also known as 4-[2-(3-Fluoro-4-trifluoromethyl-phenyl)-4-methyl-thiazol-5-ylmethylsulfanyl]-2-methyl-phenoxy}-acetic acid (CAS 317318-84-6).

GW501516 is also known as {4-[({4-methyl-2-[4-(trifluoromethyl)phenyl]-1,3-thiazol-5-yl}methyl)sulfanyl]-2-methylphenoxy}acetic acid (CAS 317318-70-0).

Pioglitazone is also known as 5-[[4-[2-(5-ethylpyridin-2-yl) ethoxy]phenyl]methyl]-1,3-thiazolidine-2,4-dione (CAS 111025-46-8).

Rosiglitazone is also known as 5-[[4-[2-[methyl(pyridin-2-yl)amino]ethoxy]phenyl]methyl]-1,3-thiazolidine-2,4-dione (CAS 155141-29-0).

MRL24 is also known as(S)-2-(3-((1-(4-Methoxybenzoyl)-2-methyl-5-(trifluoromethoxy)-1H-indol-3-yl)methyl)phenoxy)propanoic acid (CAS 393794-17-7).

9-Hydroxy-10 (E), 12 (E)-octadecadienoic acid is also known as (10E,12Z)-9-hydroxyoctadeca-10,12-dienoic acid (CAS 98524-19-7).

LY518674 is also known as 2-methyl-2-[4-[3-[1-[(4-methylphenyl)methyl]-5-oxo-4H-1,2,4-triazol-3-yl]propyl]phenoxy]propanoic acid (CAS 425671-29-0).

Hexadecanamide, its CAS number is 629-54-9.

Palmitoylethanolamide is also known as N-(2-Hydroxyethyl) hexadecanamide (CAS 544-31-0).

9-Octadecenamide, its CAS number is 3322-62-1.

GW7647 is also known as 2-[4-[2-[4-cyclohexylbutyl(cyclohexylcarbamoyl)amino]ethyl]phenyl]sulfanyl-2-methylpropanoic acid (CAS 265129-71-3).

TZD is also known as thiazolidinedione or 1,3-thiazolidine-2,4-dione (CAS 2295-31-0). Concerning 5-Hydroxy-4-phenylbutenolide, “butenolide” is also know under its IUPAC name Furan-2(5H)-one.

MTTB is also known as (E)-2-(5-((4-methoxy-2-(trifluoromethyl)quinolin-6-yl)methoxy)-2-((4-(trifluoromethyl)benzyl)oxy)-benzylidene)-hexanoic acid.

Saroglitazar, its CAS number is 495399-09-2

Elafibranor is also known as GFT505.

Lanifibranor is also known as IVA-337.

Advantageously, the composition according to the invention may further comprise a pharmaceutically acceptable excipient. The pharmaceutically acceptable excipient may be chosen depending on the route of administration of the composition.

The composition according to the invention may be administrated by any routes of administration. Routes of administration are generally classified by the location at which the substance/medicine/composition is applied. Advantageously, the composition according to the invention may be suitable for parenteral or enteral administration. Common examples include oral administration, intravenous injection, intramuscular injection, subcutaneous injection, topical administration. Preferably, the route of administration is oral administration or via a gastrointestinal probe (naso-gastric or gastrostomic procedures).

Advantageously, the composition according to the invention may be in the form of a solution (preferably an aqueous solution or a lipidic soft gel solution) or in the form of a powder (such as granulates or tablets).

Advantageously, when the composition according to the invention is a solution, the composition may comprise a solvent. The solvent may be for example water and/or an organic solvent. The solution may comprise a mixture of water and/or organic solvent(s). The solution may be reconstituted from the powder and solvent mixture comprising water and/or organic solvent(s).

Advantageously, the pharmaceutically acceptable excipient may be any excipient suitable for a pharmaceutical composition is the form of solution or powder. The pharmaceutically acceptable excipient may for example be chosen from the group comprising binders, disintegrants, fillers, dispersing agent, coating agents.

Advantageously, the composition according to the invention may comprise from 12 to 1200 mg of PPAR-modulator, preferably PPAR alpha agonists, and more preferably fibrates. The PPAR-modulator may more preferably be bezafibrate or fenofibrate.

Advantageously, the composition according to the invention may comprise from 20 to 2000 mg of urolithin derivative of formula I, preferably urolithin A.

Advantageously, the composition according to the invention may comprise from 12 to 1200 mg of PPAR-modulator, preferably PPAR alpha agonists, and more preferably fibrates, and from 20 to 2000 mg of urolithin derivative of formula I, preferably urolithin A.

Advantageously, the composition according to the invention may comprise from 20 to 2000 mg of urolithin derivative of formula I, preferably urolithin A and from 12 to 1200 mg of PPAR-modulator, preferably PPAR alpha agonists, and more preferably fibrates. Preferably, the composition according to the invention may comprise from 20 to 2000 mg of urolithin A and from 12 to 1200 mg of bezafibrate or fenofibrate, more preferably bezafibrate.

The composition of the invention may also be used as a medicine. The invention thus also encompasses the composition according to the invention for use as a medicine. The invention encompasses a therapeutic or prophylactic method of treatment comprising a step of administrating the composition according to the invention.

Advantageously, the composition according to the invention may be used in the treatment or the prevention of cardiac diseases with contributing mitochondrial dysfunction. Thus, the invention relates to a composition according to the invention for use in the treatment or the prevention of cardiac diseases with contributing mitochondrial dysfunction. The invention encompasses a therapeutic method of treatment or prevention of cardiac diseases with contributing mitochondrial dysfunction comprising a step of administrating the composition according to the invention.

It is meant by a “cardiac disease with contributing mitochondrial dysfunction” a heart disorder resulting in part from defective bioenergetics and altered mitochondrial structure and dynamics, proteostasis, and REDOX and calcium signalling. The cardiac disease may be selected from cardiac diseases with contributing mitochondrial dysfunction of primary genetic origin, cardiac diseases with contributing mitochondrial dysfunction of secondary genetic origin and cardiovascular complication caused by mitotoxic iatrogenic effects of medicines.

Advantageously, the cardiac disease with contributing mitochondrial dysfunction of primary genetic origin may be selected from MELAS (Mitochondrial Encephalomyopathy Lactic Acidosis and Stroke-like Episodes), Leigh Syndrome MERRF (Myoclonic Epilepsy and Ragged-Red Fiber Disease), MIDD (Maternally inherited diabetes-deafness syndrome), NARP (Neuropathy, Ataxia, and Retinitis Pigmentosa), GRACILE (fetal growth restriction (GR), aminoaciduria (A), cholestasis (C), iron overload (I), lactacidosis (L), and early death (E)), MNGIE (Myoneurogastointestinal Disorder and Encephalopathy), Barth Syndrome, LHON (Leber Hereditary Optic Neuropathy), Pearson Syndrome, Kearns-Sayre Syndrome, CPEO (Chronic Progressive External Ophthalmoplegia Syndrome), Freiderich's Ataxia, CoQ10 deficiency, 3-Methylglutaconic acidurias, Sengers syndrome, Wolff-Parkinson White (WPW) syndrome, Congenital cataract-hypertrophic cardiomyopathy-mitochondrial myopathy syndrome, Beta-oxidation Defects (LCAD, LCHAD, MAD, MCAD, SCAD, SCHAD, VLCAD), Carnitine-Acyl-Carnitine Deficiency, Creatine Deficiency Syndromes, Leukodystrophy, alpers disease, Polymerase Gamma (POLG) Related Disorders. Preferably, the cardiac disease with contributing mitochondrial dysfunction of primary genetic origin may be selected from the Barth syndrome and the Beta-oxidation defects.

Advantageously, the composition for use in the treatment or prevention of a cardiac disease with contributing mitochondrial dysfunction of primary genetic origin, may be administrated at the dosage range/regimen of PPAR-modulator, preferably PPAR alpha agonists, more preferably fibrates and most preferably bezafibrate or fenofibrate, of from 12 to 1200 mg/day and urolithin derivative of formula I, preferably urolithin A of from 20 to 2000 mg/day.

Advantageously, the composition for use in the treatment or prevention of a cardiac disease with contributing mitochondrial dysfunction of primary genetic origin, may be administrated at the dosage range/regimen of bezafibrate or fenofibrate, preferably bezafibrate, of from of from 12 to 1200 mg/day and urolithin A of from 20 to 2000 mg/day.

Advantageously, the cardiac diseases with contributing mitochondrial dysfunction of secondary genetic origin may be selected from the Costello Syndrome, the cardio-faciocutaneous syndrome, the neurofibromatosis type 1, the Legius syndrome, the Noonan syndrome, the Noonan syndrome with multiple lentigines and the capillary malformation-arteriovenous malformation syndrome. The cardiac diseases with contributing mitochondrial dysfunction of secondary genetic origin may preferably be the Costello syndrome.

Advantageously, the composition for use in the treatment or prevention of a cardiac disease with contributing mitochondrial dysfunction of secondary genetic origin, may be administrated at the dosage range/regimen of PPAR-modulator, preferably PPAR alpha agonists, more preferably fibrates and most preferably bezafibrate or fenofibrate of from of from 12 to 1200 mg/day and urolithin derivative of formula I, preferably urolithin A of from 20 to 2000 mg/day.

Advantageously, the composition for use in the treatment or prevention of a cardiac disease with contributing mitochondrial dysfunction of secondary genetic origin, may be administrated at the dosage range/regimen of bezafibrate or fenofibrate, preferably bezafibrate, of from of from 12 to 1200 mg/day and urolithin A of from 20 to 2000 mg/day.

Advantageously, the cardiovascular complication caused by mitotoxic iatrogenic effects of medicines may be caused by a medicine selected from alcoholism medications (such as disulfiram), analgesic and anti-inflammatory medications (such as aspirin, acetaminophen), diclofenac, fenoprofen, indomethacin, Naproxen, anesthetics (such as bupivacaine, lidocaine, propofol), angina medications (such as perhexiline), amiodarone, diethylaminoethoxyhexesterol (DEAEH), antiarrhythmic (such as amiodarone), antibiotics (such as tetracycline, antimycin A), antidepressants (such as fluoxetine, antipsychotics (such as amitriptyline, amoxapine, citalopram), chlorpromazine, fluphenazine, haloperidol, risperidone, quetiapine, clozapine, olanzapine), anxiety medications (such as alprazolam), diazepam, barbiturates (such as amobarbitalaprobarbital, butabarbital, butalbital hexobarbital, methylphenobarbital, pentobarbital, phenobarbital, primidone, propofol, secobarbital, thiobarbital), cholesterol medications, statins (such as atorvastatin, fluvastatin, lovastatin, pitavastatin, pravastatin, rosuvastatin, simvastatin), bileacids-cholestyramine, clofibrate, ciprofibrate, colestipol, colesevelam, cancer (chemotherapy) medications (such as mitomycinC, profiromycin, adriamycin (also called doxorubicin and hydroxydaunorubicin and included in the following chemotherapeuticregimens ABVD, CHOP, and FAC)), dementia medications (such as tacrine, galantamine) and diabetes medications (such as metformin, troglitazone, rosiglita-zone, buformin), HIV/AIDS medications (such as atripla). The cardiovascular complication may preferably be caused by mitotoxic iatrogenic effects of medicines selected from doxorubicin or statins.

Advantageously, the composition for use in the treatment or prevention of a cardiovascular complication caused by mitotoxic iatrogenic effects of medicines, may be administrated at the dosage range/regimen of PPAR-modulator, preferably PPAR alpha agonists, more preferably fibrates and most preferably bezafibrate or fenofibrate, of from of from 12 to 1200 mg/day and urolithin derivative of formula I, preferably urolithin A of from 20 to 2000 mg/day.

Advantageously, the composition for use in the treatment or prevention of a cardiovascular complication caused by mitotoxic iatrogenic effects of medicines, may be administrated at the dosage range/regimen of bezafibrate or fenofibrate, preferably bezafibrate, of from of from 12 to 1200 mg/day and urolithin A of from 20 to 2000 mg/day.

The composition of the invention may also be used in activation of catabolismfor supplying energy to the heart, in stimulation of the molecular machinery involved in the transduction of energy substrates through glycolysis, pyruvate oxidation and transport, TCA cycle, fatty-acid oxidation and oxidative phosphorylation for supporting heart function, and/or in activation of the hormonal system involved in the stimulation of heart function. The invention thus also encompasses the composition according to the invention for use in activation of catabolismfor supplying energy to the heart, in stimulation of the molecular machinery involved in the transduction of energy substrates through glycolysis, pyruvate oxidation and transport, TCA cycle, fatty-acid oxidation and oxidative phosphorylation for supporting heart function and/or in activation of the hormonal system involved in the stimulation of heart function. The invention encompasses a therapeutic or prophylactic method of treatment comprising a step of activation of catabolism for supplying energy to the heart, of stimulation of the molecular machinery involved in the transduction of energy substrates through glycolysis, pyruvate oxidation and transport, TCA cycle, fatty-acid oxidation and oxidative phosphorylation for supporting heart function, and/or of activation of the hormonal system involved in the stimulation of heart function.

### BRIEF DESCRIPTION OF THE FIGURES

FIG. 1 represents a combination of bezafibrate and urolithin A able to restore normal phenotype in Costello zebrafish. (A). Phenotype analysis of control (CTRL) zebrafish (i) or Costello (CS) zebrafish injected with HRASV12 plasmid (ii-vi) at 5 dpf, after 4 days of treatment (dpt) with DMSO (ii), bezafibrate (iii), urolithin A (iv) or bezafibrate (BZ) and urolithin A (UA) in combination (v-vi). Costello zebrafish developed cardiac edema (ii, iii, iv arrow), hemorrhages and vascularization defects (ii, iv star). Treatment with combination of 10 μM BZ and 5 μM UA significantly reduced the percentage of abnormal embryos (vi). (B) Description of the phenotypes observed in Costello zebrafish at 5 dpf after expression of HRASV12 plasmid. (C) Survival rate of Costello zebrafish after 4 days of treatment with DMSO, bezafibrate or urolithin A alone or in combination. Data were normalized to day 1 of treatment. (D) Percentage of defects appearance in Costello zebrafish after 4 days of treatment with DMSO, bezafibrate or urolithin A alone or in combination. (E) Expression level of the mitochondrial protein TOM20 in control or HRASG12V embryos. TOM20 protein content determined by western blot was normalized to the total protein content.

FIG. 2 represents the CS zebrafish model molecular characterization. A) Observation of the expression of the HRASV12 plasmid in embryos at the 5 days post-fertilization stage and after 4 days of treatment. Embryos from Costello zebrafish animals showing cardiac edema (A-C). Embryos from Costello zebrafish animals showing no defect after a combined treatment with bezafibrate and urolithin A at 20 μM (D-F) or 10 μM and 5 μM respectively (G-I) express the plasmid HRASV12: GFP at the same level as the embryos treated with DMSO. B) Measurement of TOM20 (mitochondrial protein marker of organelle content) was performed by western blot on whole zebrafish. Injected control or Costello animals were used and denominated as CTRL or HRASG12V respectively. The zebrafish were treated with bezafibrate (BZ) or urolithin A (UA), alone or in combination. TOM20 expression was normalized to the total protein content in the different samples.

FIG. 3 represents bezafibrate+urolithin A (BZ+UA) mode of action analysis by untargeted label-free proteomics. Whole CS zebrafish treated with BZ 10 μM+UA 5 μM were used to obtain a protein lysate and to perform an untargeted label free proteomic analysis. The data of the differential proteome were shown as a Volcano plot. The log 2fold change for each protein is shown in the abscissa (treated/untreated) and the −log 10pvalue is shown on the Y axis. The significancy threshold was set at −log p value>1.3. Data is also summarized in table 2.

FIG. 4 represents the pathway analysis of the raw data obtained from the differential proteomics study (treated versus non-treated Costello zebrafish). The differential proteomics data were analysed using IPa (Qiagen) to identify the metabolic and the signalling pathways involved in the observed rescue of the mitochondrial alterations and of the cardiac and developmental defects. The detected pathways were ranked using the activation Z-score and the significancy of the difference between the treated and the non-treated animals (−log (pvalue)).

FIG. 5 represents the efficacy of the bezafibrate+urolithin A (BZ+UA) treatment in a zebrafish model of the Barth syndrome. This model was generated using a morpholino targeting Taffazin (TAZ) and compared to a control zebrafish (CTL). (A) Survival rate was measured at day 5 post-fertilization and the toxic phenotype was determined as the % of embryos carrying one of the following defects: pericardial edema, sanguine circulation defects, low blood flux, cardiac hemorrhage, cerebral hemorrhage, necrosis, motility defects and global malformation. (B) the heartbeat rate was measured using a Kymograph on control or Barth syndrome animals at 3 dpf. (C) heartbeat rate was expressed as beat per minute (BPM) and was measured following 3 days of treatment with the BZ+UA combination at two doses in the TAZ animals. The dashed line shows the value of heartbeat rate measured in the control (CTL) animals. (D) the ejection fraction (FE) was calculated from the ventricular area determined at diastole (VTD) or systole (VTS) according to the formula: FE=(VTD−VTS)/VTD. For each measurement 19>N>30. * p<0.05, ** p<0.01, *** p<0.001.

### EXAMPLES

### Generation, Treatment and Analysis of the Zebrafish Model of the Costello Syndrome.

Zebrafish (Danio rerio) embryos were obtained from natural spawnings and raised in embryo medium (E3) (97) at 28.5° C. Embryos were staged as described previously [46]. Starting 5 days post-fertilisation (dpf), embryos were fed daily. All the studies conducted have been reviewed and approved (F341725/21063-2019061317218694). 1 nl of 12.5 ng/μl of GFP-H-RASV12 plasmid and 12.5 ng/μl of T2 transposase mRNA were co-injected in the first cell of zebrafish eggs. Control embryos were injected with the same volume of PBS. H-RASV12 was expressed in zebrafish embryo under heat shock, 30 mn at 37° C. at 1 day post fertilization (dpf) and embryo expressing GFP at 2 dpf were selected for the study.

The embryo injected with the HRASV12 plasmid were treated daily by bathing from 1 dpf to 5 dpf with respectively 20 μM bezafibrate, 20 μM urolithin A, 20 μM bezafibrate+20 μM urolithin A, 10 μM bezafibrate+5 μM urolithin A, 5 μM bezafibrate+5 μM urolithin A. Control embryos were treated with the same volume of DMSO corresponding to 0.2% of DMSO. The imaging and phenotype analysis of mortality and defects of appearance after plasmid injection were performed on alive embryo anesthetized using ethyl 3-aminobenzoate methanesulfonate, MS-222 (Sigma, Cat. #A-5040) at 200 mg/L at 2 days post fertilization, 3 dpf, 4 dpf and 5 dpf using a ZEISS axiocam stereomicroscope (ZEISS, Germany).

### Example 1: Mitochondrial Proteostasis and Bioenergetic Modulators Restore Mitochondrial Homeostasis in Costello Syndrome Cell and Animal Models

Costello syndrome is a developmental disorder caused by germline gain-of-function mutations in the HRAS/MAPK pathway. The preclinical strategy of mitochondrial stimulation was thus tested at an early stage of the disease.

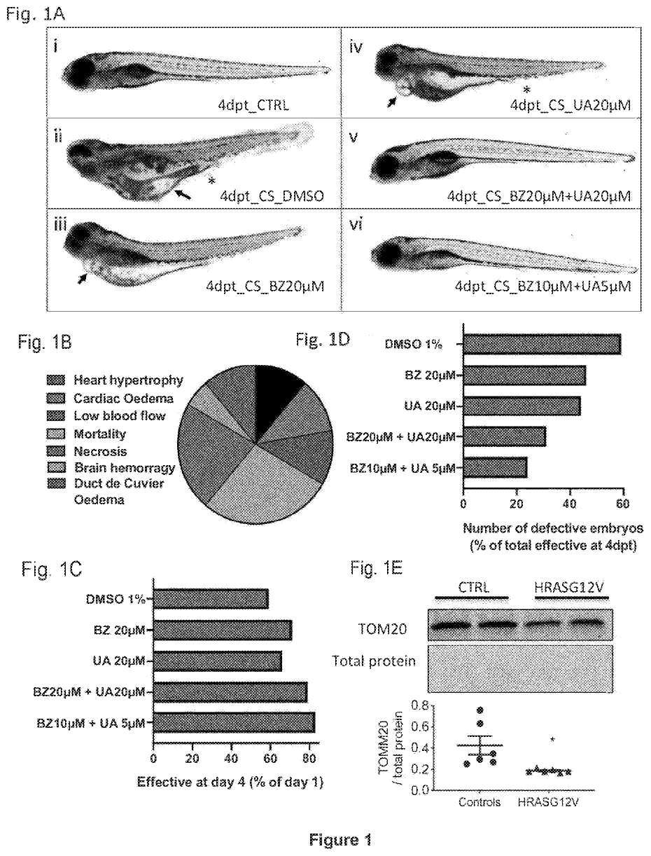

To this aim a GFP-HRASV12 zebrafish model of CS (FIG. 1) was generated, as previously described [47]. Embryos injected with HRASV12 plasmid were selected at 2 days post fertilization (dpf) following their GFP^{+/+} expression. HRASV12 plasmid was activated under a heat shock of embryos at 37° C. for 30 minutes. Embryos were observed daily from 2 dpf to 5 dpf in order to analyze the effect of HRASV12 overexpression on their physiological development (FIG. 1A,B). At 2 dpf, 22% of the HRASG12V-embryos died and this rate increased to reach 60% mortality at 5 dpf. At 5 dpf, among alive embryos, 60% presented developmental defects similar to those observed in humans and mice (FIG. 1C) as previously described [47]. A cardiac phenotype characterized by heart hypertrophy (12%), cardia and pericardia edema (12%) associated with poorly developed heart and reduced blood flow (12%) was observed. Additional phenotypes were observed including brain hemorrhage (6%) and vascularization defect leading to edema in the duct of Cuvier or malformation of the aorta or the vein in the tail region around the urogenital opening (12%) (FIG. 1A,B).

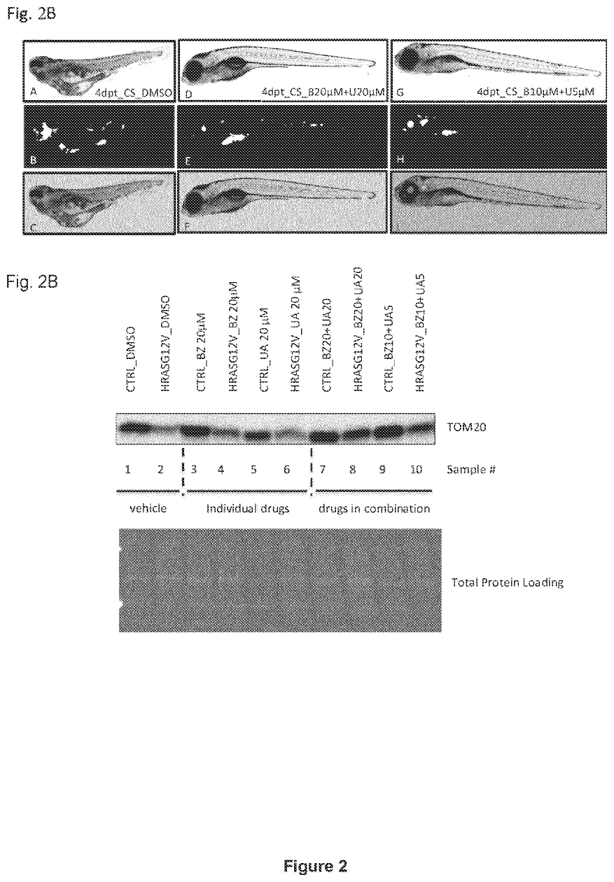

In order to analyse the impact of mitochondrial proteostasis modulators on the potential rescue of the observed HRASG12V-embryos developmental phenotype, bezafibrate and/or urolithin A were added to the zebrafish medium. Animal phenotyping revealed that the bezafibrate (BZ) or the urolithin A (UA) treatments increased the survival rate up to 70% and 65%, respectively, and decreased the number of defective individuals among alive embryo (45%, 43%) as compared to the vehicle-treated (DMSO) HRASG12V-embryos (60% of defective individuals). Interestingly, the combination of 10 μM BZ and 5 μM UA significantly increased the efficiency of the treatment by improving embryo survival up to +30% (FIG. 1C) and reduced to a large extent (3-fold) the number of animals presenting genetic developmental defects (FIG. 1D), as compared to vehicle-treated embryos. The expression of HRASV12 plasmid was verified by measuring the level GFP fluorescence emission which confirmed the stable expression of the transgene during the experiments and the absence of effect of the different treatments on plasmid expression (FIG. 2A). From 1 day post treatment (dpt) to 4 dpt, the GFP intensity remained constant in control embryos as well as in treated embryos. Last, the molecular investigation of the CS zebrafish model using western blot performed on whole embryos revealed a two-fold reduced expression of the mitochondrial marker TOM20 (FIG. 1E).

The combination treatment composed of a composition comprising 10 μM bezafibrate+5 μM urolithin A restored the expression level of TOM20 in the whole embryo and corrected the defective phenotype (FIG. 1D,E).

These preclinical findings demonstrate the efficacy of combining urolithin A and bezafibrate to restore mitochondrial proteostasis and reduce the genetic developmental defects observed in a Zebrafish model of the Costello syndrome. These original findings suggest that mitochondrial proteostasis stimulation using a combination of urolithin A and bezafibrate could be used for the treatment of rare and common diseases with mitochondrial dysfunction.

### Example 2: CS Zebrafish Model Molecular Characterization

The expression of HRASV12 plasmid was verified by measuring the level GFP fluorescence emission which confirmed the stable expression of the transgene during the experiments and the absence of effect of the different treatments on plasmid expression (FIG. 2A). The combination treatment composed of 10 μM bezafibrate+5 μM urolithin A restored the expression level of TOM20 in the whole embryo and corrected the defective phenotype (FIG. 2B).

These original findings suggest that mitochondrial proteostasis stimulation using a combination of urolithin A and bezafibrate could be used for the treatment of rare and common diseases with alteration of mitochondrial turnover.

### Example 3: Proteomic Study on Whole Zebrafish Animals, Model of the Costello Syndrome

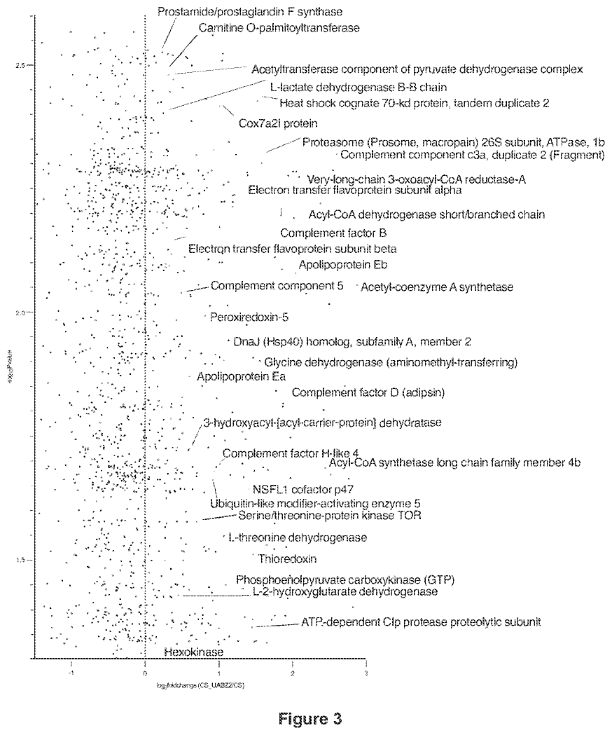

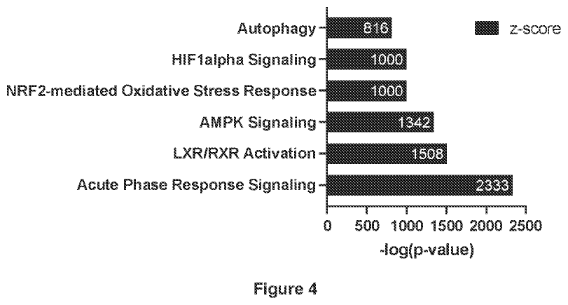

To gain more insight in the mode of action of the bezafibrate+urolithin A (BZ+UA) combination in vivo, an untargeted proteomic study on whole zebrafish animals was performed, model of the Costello syndrome, treated with the low dose (BZ 10 μM+UA 5 μM). The raw data of the proteome changes are shown as a Volcano plot (FIG. 3, table 2). For each protein, the fold change and its significancy are shown as the log 2fold change and the −log 10 (pvalue), respectively. Then, the proteins with significant change (p<0.05) were analysed using the ingenuity pathway analysis (Qiagen) database and software to identify the signalling and the metabolic pathways altered by the BZ+UA treatment (FIG. 4).

This untargeted analysis identified a mode of action of the BZ+UA treatment composed of three main mechanisms:
(1) Activation of catabolism for supplying energy to the heart,
(2) Stimulation of the molecular machinery involved in the transduction of energy substrates through oxidative phosphorylation for supporting heart function and
(3) Activation of the hormonal system involved in the stimulation of heart function.

These three components are further described below:

(1) Activation of catabolismfor supplying energy to the heart: the proteome changes revealed stimulation of autophagy, Hif1alpha signaling, NRF2-mediated oxidative stress response, AMPK signaling and LXR/RXR signaling (FIGS. 3 and 4). These pathways combine to provide energy substrates and ATP to the heart. As a result, key proteins involved in fatty-acid oxidation and glucose catabolism the main energy transducing pathways of the heart were upregulated by the BZ-UA treatment (FIG. 3). These proteins include the very-long-chain 3-oxoacyl-CoA reductase-A, short-chain fatty acids dehydrogenase, carnitine O-palmitoyltransferase, electron transfer flavoprotein subunit alpha and beta, the pyruvate carrier (MCT), the pyruvate dehydrogenase, the phosphoenolpyruvate carboxykinase, hexokinase and the glyceraldehyde 3 phosphate dehydrogenase.

This first finding on the mode of action of the BZ+UA combination suggests that the BZ+UA treatment could be used for the treatment of cardiac diseases with contributing mitochondrial dysfunction (listed above).

(2) Stimulation of the molecular machinery involved in the transduction of energy substrates through glycolysis, pyruvate oxidation and transport, TCA cycle, fatty-acid oxidation and oxidative phosphorylation: the BZ+UA treatment stimulated specific components of the mitochondrial quality control as Hsp70, Clpx, YME1-like 1b, Cox7a2, Ubiquinol-cytochrome c reductase complex assembly factor 1 and DNAJ (Hsp40 co-chaperone) as well as selected proteasome components.

This second component of the BZ+UA combination mode of action further suggests that the BZ+UA treatment could be used for the treatment of cardiac diseases with contributing mitochondrial dysfunction (listed above).

(3) Activation of the hormonal system involved in the stimulation of heart function: the changes in the proteome induced by the BZ+UA treatment in vivo, in the Costello zebrafish model, revealed activation of acute phase response signalling. This pathway describes a systemic response aiming at the restoration of tissue homeostasis.

In particular, the level of angiotensinogen (AGT), the sole precursor of all angiotensin peptides, was increased by a factor of logzfold=4 by the treatment. The angiotensin I (1-10) (produced from AGT) was also increased by logzfold=2.55. The angiotensin I (1-10) is a ligand for the G-protein coupled receptor MAS1 and has vasodilator, antidiuretic, antithrombotic and cardioprotective effects.

These molecular findings suggest using the BZ+UA combination for the treatment of cardiac diseases with contributing mitochondrial dysfunction (listed above).

Altogether, the specificity and the additivity of the three components of the BZ+UA mode of action provides a rationale to use this treatment for cardiac diseases with contributing mitochondrial dysfunction (listed above).

Altogether, the specificity of the three components of the BZ+UA mode of action provides a rationale to use a pharmaceutical composition comprising a PPAR-modulator and a compound of formula I according to the invention in a treatment for cardiac diseases with contributing mitochondrial dysfunction (listed above).

TABLE 2
List of the proteins upregulated in the CS Zebrafish by the BZ + UA treatment:

| Protein upregulated by the UA + BZ | | |
|---|---|---|
| treatment in Costello Zebrafish | log₂ foldchange | |
| (>1.5 fold with p < 0.05) | (CS_UABZ dose2/CS) | −log₁₀Pvalue |
| Serpin peptidase inhibitor, clade A (alpha-1 | 6.45925898 | 1.640996908 |
| antiproteinase, antitrypsin), member 7 | | |
| Uncharacterized protein | 4.570484456 | 1.397885521 |
| 72 kDa gelatinase | 3.716819314 | 1.631086244 |
| Chitinase | 3.479130222 | 1.852944314 |
| CCHC-type zinc finger, nucleic acid-binding | 3.179212436 | 2.274037846 |
| protein b | | |
| Acetyl-coenzyme A synthetase | 2.881464642 | 2.056611222 |
| LIM domain and actin-binding 1a | 2.819353123 | 1.405402433 |
| Zyxin | 2.767293004 | 1.665344063 |
| Glycogen [starch] synthase | 2.719989657 | 1.852377368 |
| Complement component c3a, duplicate 2 | 2.600754743 | 2.319515875 |
| (Fragment) | | |
| Angiotensin 1-10 | 2.557912671 | 2.287662747 |
| Cation-transporting ATPase | 2.521455558 | 1.673438076 |
| 60S ribosomal protein L7a | 2.499451605 | 2.017716698 |
| Acyl-CoA synthetase long chain family member 4b | 2.443424697 | 1.686876334 |
| Pre-B-cell leukemia homeobox-interacting | 2.424902414 | 1.804711706 |
| protein 1a | | |
| Serine and arginine-rich-splicing factor 9 | 2.416417363 | 1.366237129 |
| Acyl-CoA dehydrogenase short/branched chain | 2.383587222 | 2.221346521 |
| RAB7, member RAS oncogene family | 2.191923857 | 2.039068443 |
| 60S acidic ribosomal protein P2 | 2.092435448 | 2.275023611 |
| Calcium/calmodulin-dependent protein kinase | 2.055991052 | 2.285561091 |
| Apolipoprotein Eb | 2.048673523 | 2.079197903 |
| Formyltetrahydrofolate synthetase | 2.028843173 | 2.192290076 |
| Non-specific serine/threonine protein kinase | 2.01483383 | 2.286139136 |
| Beta-citrylglutamate synthase B | 1.978728804 | 1.666347132 |
| Caveolae-associated protein 2b | 1.976836735 | 1.747823916 |
| Cytochrome P450, family 2, subfamily AA, | 1.974194983 | 1.468331904 |
| polypeptide 4 | | |
| Serpin peptidase inhibitor, clade F (alpha-2 | 1.95174469 | 2.277538295 |
| antiplasmin, pigment epithelium-derived factor), | | |
| member 2b | | |
| Matrin 3-like 1.1 | 1.946594136 | 1.421879837 |
| PDZ and LIM domain 1 (elfin) | 1.918560923 | 1.526356112 |
| Cysteine and glycine-rich protein 1 | 1.912589069 | 1.387142144 |
| Elongation factor 1-beta | 1.874613082 | 1.81661063 |
| Phosphoribosyl pyrophosphate synthetase- | 1.873658201 | 1.809990292 |
| associated protein 1 | | |
| CCHC-type zinc finger, nucleic acid-binding | 1.859251037 | 2.088190422 |
| protein a | | |
| Complement factor B | 1.845601941 | 2.197204201 |
| Mitogen-activated protein kinase kinase 4A | 1.774154892 | 1.842545345 |
| Actin-related protein 2/3 complex subunit | 1.767328267 | 1.387454481 |
| Microtubule-associated protein, RP/EB family, | 1.766023011 | 2.112453951 |
| member 2 (Fragment) | | |
| Hepatocyte growth factor-regulated tyrosine | 1.762653041 | 1.574212918 |
| kinase substrate | | |
| Tight junction protein 1a | 1.753473877 | 1.522046078 |
| Anion exchange protein | 1.726268834 | 1.377856594 |
| Heterogeneous nuclear ribonucleoprotein H1, - | 1.675630575 | 1.683215485 |
| like | | |
| Fibrinogen beta chain | 1.65590287 | 1.530108072 |
| CD99 molecule | 1.648024929 | 1.333565542 |
| Transmembrane protein 214 | 1.61565562 | 1.688370487 |
| Proteasome (Prosome, macropain) 26S | 1.587751274 | 2.30162249 |
| subunit, ATPase, 1b | | |
| Glycine dehydrogenase (aminomethyl- | 1.561429393 | 1.902262738 |
| transferring) | | |
| Heat shock cognate 70-kd protein, tandem | 1.52752304 | 2.427078775 |
| duplicate 2 | | |
| Transcobalamin II | 1.507034148 | 1.337261864 |
| Far upstream element (FUSE)-binding protein 1 | 1.497613039 | 2.637355362 |
| Bridging integrator 1a (Fragment) | 1.485707621 | 1.680461715 |
| Dystrobrevin | 1.477332044 | 1.335884714 |
| Thioredoxin | 1.473645685 | 1.511256162 |
| V-type proton ATPase subunit a | 1.470622496 | 1.908936512 |
| Complement component 6 | 1.461579771 | 1.379309869 |
| ATP-dependent Clp protease proteolytic | 1.451268536 | 1.365413747 |
| subunit | | |
| NSFL1 cofactor p47 | 1.407709585 | 1.644522983 |
| Fas-associated factor family member 2 | 1.394490333 | 2.277994932 |
| Si: ch73-36611.5 | 1.391535715 | 2.616176618 |
| Cold-inducible RNA-binding protein b | 1.386786455 | 2.283375507 |
| (Fragment) | | |
| Retinol binding protein | 1.382773148 | 1.97608603 |
| Eukaryotic translation initiation factor 3 | 1.381237148 | 1.992529032 |
| subunit M | | |
| RNA helicase | 1.367763741 | 1.743896374 |
| Gamma1b-synuclein | 1.355299696 | 1.38617837 |
| Si: ch211-166a6.5 (Fragment) | 1.342228222 | 1.667729595 |
| Tubulin tyrosine ligase-like family, member 12 | 1.331833803 | 1.441999119 |
| Pbx4 homeodomain protein | 1.326547491 | 1.694813109 |
| Cytoplasmic FMR1-interacting protein | 1.325169628 | 1.759783289 |
| 5′-nucleotidase, cytosolic IB b | 1.264205562 | 1.3376038 |
| Dynactin subunit 2 | 1.235830576 | 2.318531534 |
| Valyl-tRNA synthetase | 1.222260471 | 2.237827916 |
| Formimidoyltetrahydrofolate cyclodeaminase | 1.196898582 | 2.223284274 |
| (Fragment) | | |
| LIM and SH3 domain protein 1 | 1.195470639 | 2.236403596 |
| NPC intracellular cholesterol transporter 2 | 1.192904213 | 2.213894402 |
| 2-iminobutanoate/2-iminopropanoate | 1.190947569 | 1.301164043 |
| deaminase | | |
| Amidophosphoribosyltransferase | 1.190472518 | 1.798440533 |
| Guanine nucleotide-binding protein-like 3 | 1.159108898 | 1.671943177 |
| DnaJ (Hsp40) homolog, subfamily A, member 2 | 1.14256554 | 1.94442033 |
| Neuronal cell adhesion molecule a | 1.139230705 | 2.344150936 |
| Dystrophin | 1.1391 | 1.346891136 |
| Eukaryotic translation initiation factor 5A | 1.133907888 | 2.263615154 |
| Si: ch211-183d21.1 | 1.128036375 | 2.014546011 |
| Phosphoenolpyruvate carboxykinase (GTP) | 1.097026301 | 1.450856923 |
| Cytoplasmic FMR1-interacting protein | 1.088455855 | 1.741309136 |
| Calpain inhibitor | 1.080941504 | 2.224793744 |
| Zinc finger protein 185 with LIM domain | 1.076799535 | 1.340243726 |
| L-threonine dehydrogenase | 1.076088702 | 1.548064546 |
| Signal transducer and activator of transcription | 1.073023508 | 1.904112051 |
| Cortactin | 1.067567434 | 2.257552818 |
| Microtubule-associated protein RP/EB family | 1.062381235 | 2.524221548 |
| member 1 | | |
| Serrate RNA effector molecule homolog | 1.052024792 | 2.120598717 |
| Hydroxyacid oxidase (glycolate oxidase) 1 | 1.049283556 | 2.50994522 |
| Cathepsin La | 1.047957189 | 2.102282761 |
| Serpin peptidase inhibitor, clade B (ovalbumin), | 1.044379919 | 2.223655094 |
| member 14 | | |
| Cox7a2l protein | 1.017895469 | 2.416323004 |
| Integrin, alpha 2 (CD49B, alpha 2 subunit of | 1.017315781 | 2.173325887 |
| VLA-2 receptor), tandem duplicate 2 | | |
| Zgc: 63587 | 1.016690456 | 1.909866862 |
| Complement factor H-like 4 | 1.016447607 | 1.69263306 |
| 40S ribosomal protein S17 | 1.011615533 | 2.1018881 |
| ARVCF delta catenin family member b | 1.003873878 | 1.363529842 |
| Perilipin | 0.999377087 | 1.330075581 |
| Chloride intracellular channel protein | 0.987950422 | 1.335959361 |
| Bri3-binding protein | 0.976673457 | 1.686827751 |
| Claudin | 0.97369568 | 1.828129582 |
| Dehydrogenase/reductase (SDR family) | 0.964781589 | 1.516473714 |
| member 13a, tandem duplicate 2 | | |
| Mannose receptor, C type 1a | 0.962125278 | 1.391495375 |
| Thrombospondin 1b | 0.961894784 | 1.915547328 |
| Glycine N-methyltransferase | 0.940707441 | 1.750599178 |
| 26S proteasome non-ATPase regulatory | 0.940539547 | 1.680140763 |
| subunit 4 | | |
| Calpain, small subunit 1 b | 0.936643276 | 2.327973473 |
| Ubiquitin-like modifier-activating enzyme 5 | 0.926019076 | 1.662157299 |
| Sjogren syndrome antigen B (Autoantigen La) | 0.925537117 | 2.561819587 |
| Actin-related protein 10 | 0.923701644 | 1.41804192 |
| C-1-tetrahydrofolate synthase, cytoplasmic | 0.910342615 | 1.896390279 |
| RAB5B, member RAS oncogene family | 0.906013531 | 1.435503127 |
| Epsin 2 | 0.905477045 | 2.286605117 |
| Acyl-CoA-binding domain-containing protein 6 | 0.893954772 | 1.368278758 |
| Heterogeneous nuclear ribonucleoprotein A0, - | 0.885844604 | 2.013709505 |
| like | | |
| Eukaryotic translation initiation factor 2B, | 0.871923195 | 1.521887143 |
| subunit 3 gamma | | |
| Parvalbumin 9 | 0.868354688 | 1.446264375 |
| PDZ and LIM domain 5b | 0.865408791 | 2.107489857 |
| Pre-mRNA-processing factor 8 | 0.862200368 | 1.727751159 |
| Splicing factor 1 | 0.859005878 | 1.372433447 |
| Developmentally-regulated GTP-binding | 0.854998939 | 2.28227133 |
| protein 1 | | |
| Eukaryotic translation initiation factor 5A | 0.838071453 | 2.252057978 |
| Casein kinase 2, alpha 1 polypeptide | 0.831693988 | 2.431165093 |
| Serine and arginine-rich-splicing factor 5b | 0.827627359 | 1.518204254 |
| Gamma-interferon-inducible lysosomal thiol | 0.82251645 | 1.353921028 |
| reductase | | |
| Zgc: 66479 | 0.819195597 | 1.896978329 |
| Peroxiredoxin-5 | 0.818274553 | 1.993475173 |
| Catenin (cadherin-associated protein), delta 1 | 0.809316079 | 2.246328609 |
| Uncharacterized protein | 0.807157242 | 1.636964923 |
| 3′-phosphoadenosine-5′-phosphosulfate | 0.805235686 | 1.527261581 |
| synthase | | |
| Extended synaptotagmin-like protein 1b | 0.798613752 | 2.268740758 |
| Zgc: 123103 (Fragment) | 0.792549278 | 2.199321463 |
| Galactokinase 1 | 0.784541118 | 1.404181769 |
| Twf1b protein | 0.783532014 | 1.390238606 |
| Zgc: 162509 | 0.782088498 | 2.276608053 |
| Cold-inducible RNA-binding protein a | 0.778440446 | 1.380876032 |
| Myotrophin | 0.777268113 | 1.852455891 |
| Septin 5b | 0.774435429 | 1.371579589 |
| Twinfilin actin-binding protein 1a | 0.769718247 | 1.370212727 |
| Fumarylacetoacetate hydrolase domain- | 0.762229238 | 1.91786791 |
| containing 2A | | |
| Drebrin-like b | 0.738053325 | 2.241072336 |
| C-terminal-binding protein 1 | 0.730148068 | 2.056766233 |
| LIM domain-binding 3b | 0.729289817 | 2.21550897 |
| Calcium-regulated heat-stable protein 1 | 0.721929571 | 1.380164852 |
| Ceruloplasmin | 0.721089656 | 2.286799178 |
| Calcium/calmodulin-dependent protein kinase | 0.706494738 | 1.675483099 |
| type II delta 1 chain | | |
| Serine/threonine-protein kinase TOR | 0.704238505 | 1.577417876 |
| ARD1 homolog a, N-acetyltransferase | 0.701114053 | 1.668904766 |
| Ribosomal protein S10 | 0.696768298 | 2.317332035 |
| J domain-containing protein | 0.695190812 | 1.34103678 |
| Hydroxyacid oxidase 2 (long chain) | 0.675990395 | 1.392494085 |
| Calpain, small subunit 1 a | 0.655433903 | 2.21184962 |
| ZPR1 zinc finger | 0.654272343 | 1.788156253 |
| WD repeat-containing protein 26 | 0.648156968 | 1.688687091 |
| ADP-ribosylarginine hydrolase | 0.648047875 | 2.251344434 |
| 160 kDa neurofilament protein | 0.645483922 | 1.642367704 |
| Threonyl-tRNA synthetase | 0.638348733 | 2.499853363 |
| Dynamin-type G domain-containing protein | 0.637514082 | 1.348649688 |
| Tubulin beta chain | 0.634770102 | 2.386734625 |
| Asparagine synthetase [glutamine-hydrolyzing] | 0.623471853 | 2.033507481 |
| Dihydrodiol dehydrogenase (dimeric), like | 0.62214431 | 1.348182351 |
| Obg-like ATPase 1 | 0.616932458 | 2.036466081 |
| Serine/threonine-protein phosphatase | 0.611515638 | 1.92359728 |
| Apolipoprotein Ea | 0.611198265 | 1.871170932 |
| Ubiquitin protein ligase E3 component n- | 0.609812314 | 2.236226226 |
| recognin 4 (Fragment) | | |
| THO complex 1 | 0.6065044 | 1.389791699 |
| 3-hydroxyacyl-[acyl-carrier-protein] dehydratase | 0.606069375 | 1.724865962 |
| Nucleolar protein 11-like | 0.598133209 | 1.336528122 |
| Glucosamine-6-phosphate isomerase | 0.594645616 | 1.441324149 |
| NADH-cytochrome b5 reductase | 0.591249984 | 1.722499177 |
| Very-long-chain 3-oxoacyl-CoA reductase-A | 0.585191627 | 2.235445066 |

### Example 4: CS Zebrafish Model and Barth Syndrome

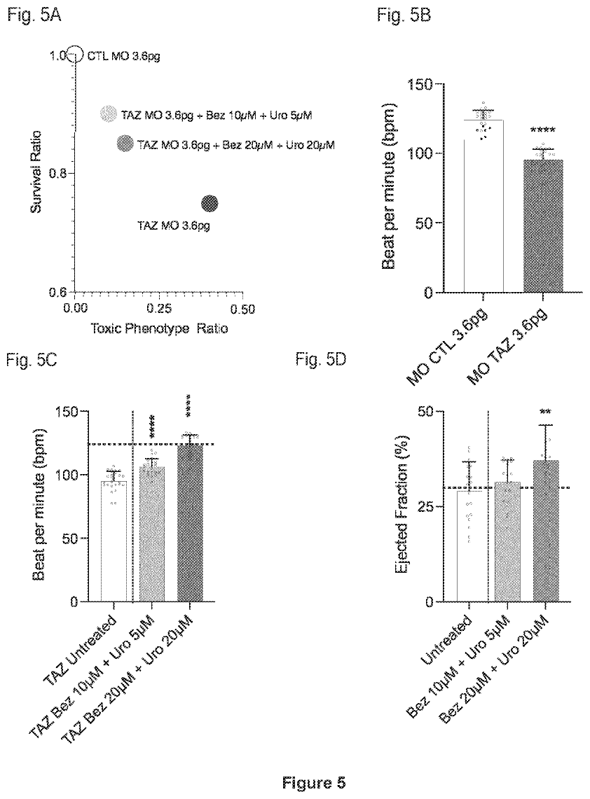

As a second example, the efficacy of the bezafibrate+urolithin A (BZ+UA) treatment in a Zebrafish model of the Barth syndrome was investigated, initially developped by Khuchua et al, 2006 [48]. This model was obtained by using a morpholino targeting the tafazzin protein (TAZ). The TAZ zebrafish has heart defects characterized by reduced heart beating rate (braddychardy) and increased ventricle. The TAZ animals have a higher mortality rate and a higher rate of phenotypic genetic abnormalities, referred to as the ‘toxic phenotype ratio’ which includes pericardic oedema, sangine circulation defects, low blood flux, cardiac hemorrhage, cerebral hemorrhage, necrosis, motility defects and global malformation (FIG. 5A). Treatment with BZ+UA at two different dosages reduced both the mortality and the penetrance of the toxic phenotype in the Barth syndrome zebrafish. The reduced heartbeat rate measured in the Barth syndrome zebrafish (TAZ) as compared to the control (CTL) is shown in FIG. 5B. Treatment with the BZ+UA combination (20 μM+20 μM) normalized the heart beating rate in the TAZ zebrafish (FIG. 5C). The ejection fraction was also increased by this treatment (FIG. 5D).

### LIST OF REFERENCES

[1] Circulation. 2019; 140:1205-1216
[2] doi:10.1016/j.bbamcr.2010.09.006/doi:10.1056/NEJMra063052/doi: 10.1172/JCI12084.
[3] Circulation. 2021; 144:1714-1731. DOI: 10.1161/CIRCULATIONAHA. 121.053575.
[4] Simanshu D K, Nissley D V., McCormick F. RAS Proteins and Their Regulators in Human Disease. Cell 2017; 170(1):17-33.
[5] Costello J. A new syndrome. N Z Med J 1971; 74:397.
[6] Aoki Y et al. Germline mutations in HRAS proto-oncogene cause Costello syndrome. [Internet]. Nat. Genet. 2005; 37(10):1038-40.
[7] Tidyman W E, Rauen K A. Noonan, Costello and cardio-facio-cutaneous syndromes: dysregulation of the Ras-MAPK pathway. Expert Rev. Mol. Med. 2008; 10.
[8] Gibbs J B, Sigal I S, Poe M, Scolnick E M. Intrinsic GTPase activity distinguishes normal and oncogenic ras p21 molecules. Proc. Natl. Acad. Sci. U.S.A 1984; 81(18):5704-5708.
[9] Siegel D H, Mann J A, Krol A L, Rauen K A. Dermatological phenotype in Costello syndrome: consequences of Ras dysregulation in development: Dermatological phenotype and Ras dysregulation in Costello syndrome. Br. J. Dermatol. 2012; 166(3):601-607.
[10] Gripp K W. Tumor predisposition in Costello syndrome. Am. J. Med. Genet. Part C Semin. Med. Genet. 2005; 137C(1):72-77.
[11] Estep A L, Tidyman W E, Teitell M A, Cotter P D, Rauen K A. HRAS mutations in Costello syndrome: detection of constitutional activating mutations in codon 12 and 13 and loss of wild-type allele in malignancy. Am. J. Med. Genet. A 2006; 140(1):8-16.
[12] Lin A E et al. Clinical, pathological, and molecular analyses of cardiovascular abnormalities in Costello syndrome: A Ras/MAPK pathway syndrome. Am. J. Med. Genet. Part A 2011; 155(3):486-507.
[13] Siwik E S, Zahka K G, Wiesner G L, Limwongse C. Cardiac disease in Costello syndrome. [Internet]. Pediatrics 1998; 101(4 Pt 1):706-9.
[14] Wei B-R et al. Capacity for resolution of Ras-MAPK-initiated early pathogenic myocardial hypertrophy modeled in mice. [Internet]. Comp. Med. 2011; 61(2):109-18.
[15] Hunter J J, Tanaka N, Rockman H A, Ross J, Chien K R. Ventricular expression of a MLC-2v-ras fusion gene induces cardiac hypertrophy and selective diastolic dysfunction in transgenic mice. [Internet]. J. Biol. Chem. 1995; 270(39):23173-8.
[16] Schuhmacher A J et al. A mouse model for Costello syndrome reveals an Ang Il-mediated hypertensive condition. [Internet]. J. Clin. Invest. 2008; 118(6):2169-79.
[17] Gottshall K R et al. Ras-dependent pathways induce obstructive hypertrophy in echo-selected transgenic mice. [Internet]. Proc. Natl. Acad. Sci. U.S.A 1997; 94(9):4710-5.
[18] Sana M E et al. A Novel HRAS Mutation Independently Contributes to Left Ventricular Hypertrophy in a Family with a Known MYH7 Mutation. [Internet]. PLOS One 2016; 11(12):e0168501.
[19] Oba D et al. Mice with an Oncogenic HRAS Mutation are Resistant to High-Fat Diet-Induced Obesity and Exhibit Impaired Hepatic Energy Homeostasis. [Internet]. EBioMedicine 2018; 27:138-150.
[20] Rauen K. HRAS and the Costello syndrome. Clin. Genet. 2007; 71(2):101-108.
[21] Gripp K W, Lin A E. Costello syndrome: a Ras/mitogen activated protein kinase pathway syndrome (rasopathy) resulting from HRAS germline mutations. Genet. Med. 2012; 14(3):285-292.
[22] Hakim K, Boussaada R, Hamdi I, Msaad H. Cardiac events in Costello syndrome: One case and a review of the literature. [Internet]. J. Saudi Hear. Assoc. 2014; 26(2):105-9.
[23] Ormerod J O M, Frenneaux M P, Sherrid M V. Myocardial energy depletion and dynamic systolic dysfunction in hypertrophic cardiomyopathy [Internet]. Nat. Rev. Cardiol. 2016; 13(11):677-687.
[24] Kloner R A et al. New and revisited approaches to preserving the reperfused myocardium. [Internet]. Nat. Rev. Cardiol. 2017; 14(11):679-693.
[25] McManus M J et al. Mitochondrial DNA Variation Dictates Expressivity and Progression of Nuclear DNA Mutations Causing Cardiomyopathy. [Internet]. Cell Metab. [published online ahead of print: Aug. 23, 2018]; doi:10.1016/j.cmet.2018.08.002.
[26] Murphy E et al. Mitochondrial Function, Biology, and Role in Disease [Internet]. Circ. Res. 2016; 118(12):1960-1991.
[27] Kleefstra T et al. Mitochondrial dysfunction and organic aciduria in five patients carrying mutations in the Ras-MAPK pathway. [Internet]. Eur. J. Hum. Genet. 2011; 19(2):138-44.
[28] Dard L, Bellance N, Lacombe D, Rossignol R. RAS signalling in energy metabolism and rare human diseases. Biochim. Biophys. Acta-Bioenerg. [published online ahead of print: 2018]; doi:10.1016/j.bbabio.2018.05.003.
[29] Starling E H, Visscher M B. The regulation of the energy output of the heart. J. Physiol. 1927; 62(3):243-261.
[30] Prebble J N. The discovery of oxidative phosphorylation: a conceptual off-shoot from the study of glycolysis. Stud. Hist. Philos. Sci. Part C Stud. Hist. Philos. Biol. Biomed. Sci. 2010; 41(3):253-262.
[31] Arany Z et al. Transcriptional coactivator PGC-1α controls the energy state and contractile function of cardiac muscle [Internet]. Cell Metab. 2005; 1(4):259-271.
[32] Kärkkäinen O et al. Heart specific PGC-1α deletion identifies metabolome of cardiac restricted metabolic heart failure [Internet]. Cardiovasc. Res. [published online ahead of print: Jun. 20, 2018]; doi:10.1093/cvr/cvy155.
[33] (Nucleic Acids Research, Volume 49, Issue D1, 8 Jan. 2021, Pages D1541-D1547, https://doi.org/10.1093/nar/gkaa1011).
[34] Rowe G C, Jiang A, Arany Z. PGC-1 coactivators in cardiac development and disease. [Internet]. Circ. Res. 2010; 107(7):825-38.
[35] Fan J et al. Autophagy as a Potential Target for Sarcopenia. [Internet]. J. Cell. Physiol. 2016; 231(7):1450-9.
[36] Bujak A L et al. AMPK Activation of Muscle Autophagy Prevents Fasting-Induced Hypoglycemia and Myopathy during Aging [Internet]. Cell Metab. 2015; 21(6):883-890.
[37] Hu X et al. AMP Activated Protein Kinase-2 Regulates Expression of Estrogen-Related Receptor-, a Metabolic Transcription Factor Related to Heart Failure Development [Internet]. Hypertension 2011; 58(4):696-703.
[38] Erkens R et al. Left ventricular diastolic dysfunction in Nrf2 knock out mice is associated with cardiac hypertrophy, decreased expression of SERCA2a, and preserved endothelial function [Internet]. Free Radic. Biol. Med. 2015; 89:906-917.
[39] Huss J M et al. The nuclear receptor ERRalpha is required for the bioenergetic and functional adaptation to cardiac pressure overload. [Internet]. Cell Metab. 2007; 6(1):25-37.
[40] Luptak I et al. Decreased contractile and metabolic reserve in peroxisome proliferator-activated receptor-alpha-null hearts can be rescued by increasing glucose transport and utilization. [Internet]. Circulation 2005; 112(15):2339-46.
[41] Fan Y et al. Resveratrol Ameliorates Cardiac Hypertrophy by Down-regulation of miR-155 Through Activation of Breast Cancer Type 1 Susceptibility Protein. [Internet]. J. Am. Heart Assoc. 2016; 5(4). doi:10.1161/JAHA. 115.002648.
[42] Meng R-S et al. Adenosine monophosphate-activated protein kinase inhibits cardiac hypertrophy through reactivating peroxisome proliferator-activated receptor-α signaling pathway [Internet]. Eur. J. Pharmacol. 2009; 620(1-3):63-70. Thandapilly S J et al. Resveratrol Prevents the Development of Pathological
[43] Cardiac Hypertrophy and Contractile Dysfunction in the SHR Without Lowering Blood Pressure [Internet]. Am. J. Hypertens. 2010; 23(2):192-196.
[44] Huang Y et al. The PPAR pan-agonist bezafibrate ameliorates cardiomyopathy in a mouse model of Barth syndrome [Internet]. Orphanet J. Rare Dis. 2017; 12(1):49.
[45] Takada & Makoto Makishima (2020), a patent review (2014-present), Expert Opinion on Therapeutic Patents, 30:1, 1-13, DOI: 10.1080/13543776.2020.1703952;
[46] Kimmel C B, Ballard W W, Kimmel S R, Ullmann B, Schilling T F. Stages of embryonic development of the zebrafish. [Internet]. Dev. Dyn. 1995; 203(3):253-310.
[47] Santoriello C et al. Expression of H-RASV12 in a zebrafish model of Costello syndrome causes cellular senescence in adult proliferating cells [Internet]. Dis Model. Mech. 2009; 2(1-2):56-67.
[48] Zaza Khuchua, Zou Yue, Lorene Batts, Arnold W Strauss A zebrafish model of human Barth syndrome reveals the essential role of tafazzin in cardiac development and function. Circ Res. 2006 Jul. 21; 99(2):201-8. doi: 10.1161/01.RES.0000233378.95325.ce.

1. A pharmaceutical composition comprising a PPAR-modulator and a compound of formula I: 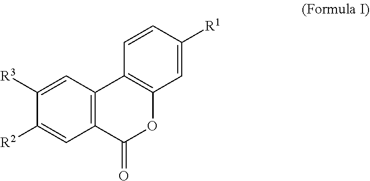
wherein R¹, R² and R³ independently represents H or OH.

2. The composition according to claim 1, wherein the compound of formula I is urolithin A, B and/or C, preferably urolithin A.

3. The composition according to claim 1, wherein the PPAR-modulator is chosen in the group comprising PPAR alpha agonists, PPAR gamma agonists, PPAR delta agonists, PPAR dual agonists (alpha/gamma or alpha/delta) and PPAR pan agonists (alpha/gamma/delta).

4. The composition according to claim 1, wherein the PPAR-modulator is bezafibrate or fenofibrate, preferably bezafibrate.

5. The composition according to claim 1, further comprising a pharmaceutically acceptable excipient.

6. The composition according to claim 1, comprising from 12 to 1200 mg of PPAR-modulator.

7. The composition according to claim 1, comprising from 20 to 2000 mg of compound of formula I.

8. The composition according to claim 1 for use as a medicine.

9. The composition according to claim 1 for use in the treatment or prevention of cardiac diseases with contributing mitochondrial dysfunction.

10. The composition for use according to claim 9, wherein the cardiac disease is selected from cardiac diseases with contributing mitochondrial dysfunction of primary genetic origin, cardiac diseases with contributing mitochondrial dysfunction of secondary genetic origin and cardiovascular complication caused by mitotoxic iatrogenic effects of medicines.

11. The composition for use according to claim 10, wherein the cardiac disease with contributing mitochondrial dysfunction of primary genetic origin is the Barth syndrome.

12. The composition for use according to claim 10, wherein the cardiac diseases with contributing mitochondrial dysfunction of secondary genetic origin is the Costello syndrome.

13. The composition for use according to claim 10, wherein the cardiac disease is a cardiac disease with contributing mitochondrial dysfunction of primary genetic origin and wherein the composition is administrable at a dosage range/regimen of PPAR-modulator, preferably PPAR alpha agonists, more preferably fibrates and most preferably bezafibrate or fenofibrate, of from of from 12 to 1200 mg/day and compound of formula I, preferably urolithin A, of from 20 to 2000 mg/day.

14. The composition for use according to claim 10, wherein the cardiac disease is a cardiac disease with contributing mitochondrial dysfunction of secondary genetic origin and wherein the composition is administrable at a dosage range/regimen of PPAR-modulator, preferably PPAR alpha agonists, more preferably fibrates and most preferably bezafibrate or fenofibrate, of from of from 12 to 1200 mg/day and compound of formula I, preferably urolithin A, of from 20 to 2000 mg/day.

15. The composition for use according to claim 10, wherein the cardiac disease is a cardiac disease caused is a cardiovascular complication caused by mitotoxic iatrogenic effects of medicines and wherein the composition is administrable at a dosage range/regimen of PPAR-modulator, preferably PPAR alpha agonists, more preferably fibrates and most preferably bezafibrate or fenofibrate, of from of from 12 to 1200 mg/day and compound of formula I, preferably urolithin A, of from 20 to 2000 mg/day.

16. The composition according to claim 1 for use in activation of catabolism for supplying energy to the heart, for use in stimulation of the molecular machinery involved in the transduction of energy substrates through glycolysis, pyruvate oxidation and transport, TCA cycle, fatty-acid oxidation and oxidative phosphorylation for supporting heart function and/or for use in activation of the hormonal system involved in the stimulation of heart function.
